(19) United States
(12) Patent Application Publication
(10) Pub. No.: US 2011/0172304 A1
(43) Pub. Date: Jul. 14, 2011

(54) Sex Attractant of White Peach Scale and Attraction Method

(75) Inventors: Naoki Ishibashi, Joetsu-shi (JP); Minoru Nakamura, Joetsu-shi (JP); Fumiaki Mochizuki, Joetsu-shi (JP); Takehiko Fukumoto, Joetsu-shi (JP)

(73) Assignee: Shin-Etsu Chemical Co., Ltd.

(21) Appl. No.: 13/004,364

(22) Filed: Jan. 11, 2011

(30) Foreign Application Priority Data
Jan. 12, 2010 (JP) 2010-004214

(51) Int. Cl. A01N 37/02; C07C 69/24; A01P 19/00

(52) U.S. Cl. 514/546; 560/261

(57) ABSTRACT

Provided is an attractant of white peach scale capable of simplifying the synthesis of a sex pheromone of white peach scale and suppressing attraction of a parasitoid wasp which is a natural enemy of the white peach scale. More specifically, provided is a sex attractant of white peach scale comprising at least (R,Z)-(+)-3,9-dimethyl-6-isopropenyl-3,9-decadienyl priopionate and a geometric isomer (R,E)-(+)-3,9-dimethyl-6-isopropenyl-3,9-decadienyl priopionate at a weight ratio ranging from 80:20 to 40:60. In addition, provided is a method for attracting white peach scale by using said attractant.

### CROSS-RELATED APPLICATIONS

This application claims priority from Japanese Patent Application No. 2010-004214; filed Jan. 12, 2010, the disclosure of which is incorporated herein by reference in its entirety.

### BACKGROUND OF THE INVENTION

1. Field of the Invention

The present invention relates to an attractant and an attraction method using a synthetic pheromone of a white peach scale (scientific name: Pseudaulacaspis pnetagona) which is an insect pest of tea.

2. Description of the Related Art

The white peach scales are insect pests firmly attached to branches or trunks of tea trees and fed thereby. They are distributed in the tea production areas throughout the country. In tea cultivation, so-called “collar pruning”, that is, pruning of tea trees into about half of their height is performed periodically in order to rejuvenate the trees and obtain new shoots having a high quality. In the collar pruning, the operation itself is a hard work and moreover, new leaves cannot be harvested for several months after collar pruning. In usual tea cultivation, the collar pruning is said to be conducted at intervals of from five to seven years.

However, tea trees damaged with white peach scales need the collar pruning at intervals of about four years due to a decline in tree vigor earlier than expected. This becomes a serious trouble in tea cultivation. Although spraying of an insecticide is only one control method, an insecticide solution does not reach white peach scales. It is because white peach scales are in the adult female scale when they are eggs, while they protect their bodies with wax made by themselves when they are from the second-instar larvae to pupae. Accordingly, it is very difficult to control them by using an insecticide. They get out from their scales in order to move to another place only for the periods of the first-instar larvae that have hatched from eggs and adult males that have emerged from the pupae. These periods are suited for the control of this insect pest. It is therefore very important to predict these periods for controlling this insect pest.

A method of estimating an emergence period of the first-instar larvae based on the degree-day has been studied. However, a mathematical formula that is true at one place is often not applicable to a district, a field, or a generation different therefrom so that there is no established technique.

In recent years, the chemical structure of a sex pheromone substance of many moths has been identified. In general, a sex pheromone substance is a chemical substance extracted from adult females and even miniscule quantity of it strongly attracts adult males of the same species. A pheromone trap uses, as an attractant, such a pheromone obtained by chemically synthesis. When the number of insect pests caught by a pheromone trap is counted at regular intervals, it is possible to know, as timely information, the time and amount of emergence of the insect pests in a target field. This makes it possible to control the insect pests at the optimum time. Currently, pheromone traps are utilized for emergence prediction in many lepidopterous insect pests.

There is another method for utilizing a synthetic pheromone. It is a method of making use of a strong attractiveness of a synthetic pheromone, thereby catching many males living in a farm and directly reducing a pest density. This method is called a mass trapping method and has been put into practical use in trapping of lepidopterous or coleopterous insect pests. In recent years, there has been reported, in many lepidopterous insects, a mating disruption method of spraying a large amount of a synthetic pheromone in a field to disrupt a mating behavior and reducing the density of next-generation insect pests.

It is reported that the sex pheromone of white peach scales is (R,Z)-(+)-3,9-dimethyl-6-isopropenyl-3,9-decadienyl propionate (J. Chem. Ecol. 941-953 (1979)). A process of synthesizing this compound is also reported (J. Org. Chem. 1980, 45, 2910-2913). It has already been known that Thomsonisca amathus which is a parasitoid wasp and a strong natural enemy of white peach scale is attracted simultaneously to the synthetic sex pheromone of white peach scale (Kunihiko Matsuhira and Yasunari Kozaki, Bulletin of the Kagoshima Tea Experiment Station 15 (2001)).

### SUMMARY OF THE INVENTION

The present invention is made with the forgoing in view. In particular, an object of the invention is to provide an attractant and an attraction method of white peach scale which is expected to be utilized in emergence prediction or a mass trapping method.

Utilization of a sex attractant of white peach scale has two problems. One is a very complex structure of synthetic sex pheromone itself used as a sex attractant (J. Chem. Ecol. 941-953 (1979)), which makes synthesis and purification of it cumbersome. The sex pheromone of white peach scale is, as described above, (R,Z)-(+)-3,9-dimethyl-6-isopropenyl-3,9-decadienyl propionate and the (Z) isomer of it has activity. The synthesis process described in J. Org. Chem. 1980, 45, 2910-2913 is not only cumbersome but also requires purification because due to the presence of a double bond, mixture of a geometric isomer is inevitable because of the characteristics of the synthesis process. Accordingly, preparation of an intended synthetic sex pheromone needs cumbersome synthesis and purification operations and therefore requires a huge amount of labor, which poses a great problem.

The other problem is that, as described in Kunihiko Matsuhira and Yasunari Kozaki, Bulletin of the Kagoshima Tea Experiment Station 15 (2001), Thomsonisca amathus which is a parasitoid wasp and a strong natural enemy of white peach scale is attracted simultaneously to the synthetic sex pheromone of white peach scale. The cause for it was not elucidated before.

Both the white peach scale and the natural-enemy parasitoid wasp have body sizes smaller than 1 mm so that when they are captured on a trap, they cannot usually be discriminated from each other visually. For discrimination, a loupe or a stereoscopic microscope of a high magnification is necessary. A large number of natural-enemy parasitoid wasps captured on the trap make the research of white peach scale more cumbersome. In addition, because the white peach scale cannot be controlled easily with an insecticide as described above, an indigenous natural enemy having an influence on it should be protected as much as possible. Attraction of a large number of natural-enemy parasitoid wasps is not desired also from the standpoint of biological control making use of natural enemy.

The present invention provides an attractant of white peach scale capable of simplifying the synthesis of a sex pheromone of white peach scale and suppressing attraction of a natural-enemy parasitoid wasp.

The present inventors have carried out an extensive investigation with a view to overcoming the above problems. As a result, it has been found that when the content of the E isomer is from 20 to 60% by weight, preferably from 30 to 50% by weight, the number of Thomsonisca amathus which is a natural-enemy parasitoid wasp can be reduced by 30% or greater without drastically reducing the attraction number of white peach scales, leading to the present invention.

According to the present invention, provided is a sex attractant of white peach scale comprising (R,Z)-(+)-3,9-dimethyl-6-isopropenyl-3,9-decadienyl propionate and a geometric isomer (R,E)-(+)-3,9-dimethyl-6-isopropenyl-3,9-decadienyl propionate at a weight ratio of from 80:20 to 40:60. Also is provided an method for attracting the white peach scale by using this attractant.

According to the present invention, a synthetic sex pheromone of white peach scale in the presence of a geometric isomer available by using a known organic synthesis technique can be used without any particular cumbersome operation for synthesis and purification. Moreover, an attractant and an attraction method facilitating research in emergence prediction and capable of protecting a natural-enemy parasitoid wasp as much as possible in emergence prediction and mass trapping can be obtained by reducing the trapping number of Thomsonisca amathus which is a parasitoid wasp and a strong natural enemy of white peach scale.

### DETAILED DESCRIPTION OF THE PREFERRED EMBODIMENTS

The sex attraction substance of white peach scale is (R,Z)-(+)-3,9-dimethyl-6-isopropenyl-3,9-decadienyl propionate. It can be synthesized in a known manner.

For example, according to the synthesis process described in J. Org. Chem. 1980, 45, 2910-2913, a mixture of the Z and E isomers is obtained by reacting an alcohol, which is produced by reaction of (R)-(+)-6-methyl-3-isopropenyl-6-heptenal with phosphorane prepared from ethyl triphenylphosphonium bromide, ethylene oxide and n-butyl lithium, with propionyl chloride, and then separated into the Z isomer and the E isomer by using liquid column chromatography or the like. In the present invention, on the other hand, separation into the Z isomer and the E isomer is not necessary so that a separating operation such as liquid column chromatography can be omitted, leading to release from a cumbersome purification operation.

After separation of the sex attraction substance thus obtained into the Z isomer and the E isomer by using liquid column chromatography, nine attractants containing a (Z isomer):(E isomer) weight ratio of 100:0, 90:10, 80:20, 70:30, 60:40, 50:50, 40:60, 20:80 and 0:100 were prepared, respectively. Comparison in attraction property between the white peach scale and Thomsonisca amathus which is a parasitoid wasp and natural enemy thereof has revealed that the (Z isomer):(E isomer) weight ratio is preferably within a range of from 80:20 to 40:60, more preferably within a range of from 70:30 to 50:50.

Although there is no particular limitation on a container having therein the sex attractant of white peach scale, a rubber cap may be preferred. The total amount of the Z and E isomers contained in the container is preferably from 0.01 to 10 mg.

When the attractant is utilized for emergence prediction, it is preferable to constantly place one or two traps per hectare for five months from May to September when adult insects emerge. As for a preferable amount of the sex attraction substance, 0.01 to 0.1 mg of a total amount of the Z and E isomers can make monitoring possible. Increasing the amount of the sex attraction substance may make the research complicated because of an increase in the number of traps. On the other hand, when the attractant is used for mass trapping, it is preferable to place at least ten traps per hectare for two months during the period of emergence of the above adult insects. The greater the number of insects captured by the trap, the higher the control effect. As for the preferable amount of the sex attraction substance, 0.5 to 10 mg of a total amount of the Z and E isomers may be desirable.

### EXAMPLES

The embodiments for the present invention will next be described by using Examples and Comparative Examples, but it should not be construed that the present invention is limited to or by these Examples.

### (1) Test Material

In accordance with the process described in J. Org. Chem. 1980, 45, 2910-2913, (R,Z)-(+)-3,9-dimethyl-6-isopropenyl-3,9-decadienyl propionate, the sex pheromone of white peach scale, was synthesized. The product thus obtained was separated into the Z isomer and the E isomer by using medium-pressure liquid column chromatography. A gray rubber cap (produced by West in US) impregnated with 0.1 mg in total of the Z isomer and the E isomer at a weight ratio of 100:0, 90:10, 80:20, 70:30, 60:40, 50:50, 40:60, 20:80 or 0:100 was used as an attractant for test.

### (2) Survey

In the emerging season of first-generation adult males of white peach scale in June, 2007, the attractant described above was attached to a white sticky trap and placed at intervals of 10 m on ridges in a tea field. The numbers of adult males of white peach scale and Thomsonisca amathus, a parasitoid wasp and natural enemy thereof, which had stuck to the sticky plates, was counted every day, respectively.

### (3) Data Processing

When the number of adult males of white peach scale attracted to the trap in Comparative Example 1 comprising an attractant containing 100% of (R,Z)-(+)-3,9-dimethyl-6-isopropenyl-3,9-decadienyl propionate was assigned as 100, the number attracted to each trap was calculated as a relative value. In the attraction test of Thomsonisca amathus, similar data processing was performed.

### (4) Results

In Examples 1 to 5 in which the (Z isomer):(E isomer) weight ratio was adjusted to fall in a range of from 80:20 to 40:60, an attraction property of 92.8 to 33.3% was secured when compared with the attractant in Comparative Example 1 containing 100% by weight of the Z isomer reported as the sex pheromone of white peach scale. The weight ratio of 20:80 in Comparative Example 3 resulted in the attraction property as low as 15% or less.

The attraction property of Thomsonisca amathus also decreases due to an increase in the content of the E isomer. It decreases drastically. When the content of the E isomer is adjusted to 20% or greater, the number of Thomsonisca amathus thus attracted is 70% or less.

The above results have revealed that by adjusting the (Z isomer):(E isomer) weight ratio to fall within a range of from 80:20 to 40:60, preferably from 70:30 to 50:50, it becomes possible to reduce the attracted number of Thomsonisca amathus, a natural-enemy parasitoid wasp, without drastically deteriorating the attraction property of white peach scale.

TABLE 1

| | | | attraction of | attraction of |
|---|---|---|---|---|
| | content | | adult males of | Thomsonisca |
| | Z isomer | E isomer | white peach scale | amathus |
| | (wt %) | (wt %) | (%) | (%) |
| Example 1 | 80 | 20 | 92.8 | 69.5 |
| Example 2 | 70 | 30 | 84.2 | 54.1 |
| Example 3 | 60 | 40 | 80.7 | 43.8 |
| Example 4 | 50 | 50 | 74.0 | 21.4 |
| Example 5 | 40 | 60 | 33.3 | 12.3 |
| Comp.Ex. 1 | 100 | 0 | 100.0 | 100.0 |
| Comp.Ex. 2 | 90 | 10 | 96.2 | 90.1 |
| Comp.Ex. 3 | 20 | 80 | 14.3 | 8.9 |
| Comp.Ex. 4 | 0 | 100 | 0.0 | 0.0 |

1. A sex attractant of white peach scale, comprising at least (R,Z)-(+)-3,9-dimethyl-6-isopropenyl-3,9-decadienyl priopionate and a geometric isomer (R,E)-(+)-3,9-dimethyl-6-isopropenyl-3,9-decadienyl priopionate at a weight ratio ranging from 80:20 to 40:60.

2. A method for attracting white peach scale, comprising placing the attractant as claimed in claim 1 in a tea field.
